(19) United States
(12) Patent Application Publication
(10) Pub. No.: US 2021/0145709 A1
(43) Pub. Date: May 20, 2021

(54) PRISTINE GRAPHENE AND COMPOSITIONS WITH NATURAL PRESSURE SENSITIVE ADHESIVE (PSA) METHOD FOR OVER SKIN APPLICATIONS

(71) Applicant: BIO-NATURE LABORATORIES LLC, Edison, NJ (US)

(72) Inventors: Michael Kruel, Flemington, NJ (US); Lily Kruel, Flemington, NJ (US)

(73) Assignee: BIO-NATURE LABORATORIES LLC, Edison, NJ (US)

(21) Appl. No.: 17/052,897

(22) PCT Filed: May 6, 2019
(86) PCT No.: PCT/US19/30898

§ 371 Date: Nov. 4, 2020

Related U.S. Application Data
(60) Provisional application No. 62/667,121, filed on May 4, 2018.

(51) Int. Cl. A61K 8/19; A61K 8/895; A61Q 19/08

(52) U.S. Cl. CPC A61K 8/19; A61K 8/895; A61K 2800/884; A61K 2800/95; A61K 2800/31; A61Q 19/08

(57) ABSTRACT

This patent document discloses a kit containing abase component and an activating component for cosmetics, therapeutic and aesthetic application purposes. The kit, comprised innovative technology, ingredients, and formulations that overcome the drawbacks of unviable, unpractical, unwearable and unmarketable products, enables a film-formed “young-looking skin” to cling onto the real skin for much longer period of time, and is truly wearable and viable to the consumers for achieving the benefits of dramatically reducing or diminishing eye bags, dark circles, enlarged pores, wrinkles, age spots, and so on with desired and rapid results. The film is comfortable, invisible, safe, non-invasive and easy to wear.

### FIELD OF THE INVENTION

This current invention discloses a novel kit and film for cosmetics, make-up, therapeutic and aesthetic application purpose.

### BACKGROUND

Under eye bags, puffiness, dark circles, wrinkles, enlarged pores and age spots are the problems that earned while we are in the aging process. It happens to almost everyone on this planet, some more and some less, due to hereditary, facial bone structure or living habits. Those people who have heavily fat-deposited sagging bags feel and look tired, and some have distorted faces due to the eye bags hanging down. Because of these symptoms, many people lost confidence and desire to appear in the public events. They are not happy with the way how they look and feel!

Some people rely on skin care and cosmetics products such as BB/CC cream, concealer or foundation to hide their appearance, but the results are minimal and insufficient. Some cosmetic products may make the appearance even worse, due to powders/creams siting in between the cracks and creases of the skin, making the wearer look older and skin blemishes and eye bags stand out more prominently.

Some people turn to surgical measures, such as facial surgeries to tighten the sagging skin or remove eye bags, laser treatment for age spots, and products such as Botox® as the filler for the wrinkles, etc. These are dramatic measures, and they are short-term fixes, sometimes with serious side effects. These procedures are more costly with long-term health concerns, e.g. permanent facial muscle damage, and surgeries cannot be undone without more surgery. Therefore, so many people just gave up!

There are some companies/organizations that attempted to use large molecule ingredients or silicone elastomer compounds to make skin film-forming products in an effort to temporarily correct the appearance of signs of aging. But due to the silicones' mechanical limitations and the nature forces of human skin movement, the products were not viable to the general consumers to wear, especially in water based formulations. Prior products claiming to provide a film-forming layer of skin have been unsuccessful, some being pulled off the market entirely. One shortcoming with these alternative products is that the edges of the film start to peel off within a few hours. Additionally, there are many restrictions for such products, e.g. no water/moisture contact, no tearing, etc. A need exists for a viable, wearable technology, ingredients and formulations that overcome the above drawbacks.

### SUMMARY

With this invention, the anhydrous proprietary compositions, used graphene for strength and a natural pressure-sensitive adhesive (PSA), which enhanced and maximized the mechanical functionality and performance, resulting in a superior, wearable, and viable natural-looking cosmetic “young-looking skin” film that is easy to wear without the difficulties, drawbacks, and restrictions of prior film-forming compositions. These novel compositions and natural PSA provide a stronger, more durable, more flexible, more breathable, and adhesively bonded final beauty product. This composition enables the film-formed “young-looking skin” to cling onto the real skin and is truly wearable and viable to the consumers for achieving the benefits of dramatically reducing or diminishing eye bags, dark circles, enlarged pores, wrinkles, age spots, and so on with designed results. Now with this invention, these products can be easily worn for 24 or more hours and can perform and maintain daily normal living activities during the wearing period. They are comfortable, invisible, safe, and non-invasive.

An aspect of the invention provides a kit for improving skin aesthetic, comprising (a) a base component comprising graphene and a cross-linkable elastomer; and (b) an activating component comprising an agent promoting the cross-linking of the elastomer.

The graphene ranges from about 0.01% to about 30% by weight in the base component. In some embodiments, the graphene ranges from about 0.04% to about 10% by weight in the base component. The graphene is preferably processed with microwave, vacuum, ultrasonication, or centrifuge before use. In some embodiments, the base component is substantially free from water. In some embodiments, the base component and/or the activating component are substantially free from water. In some embodiments, the base component is substantially free from water and contains one or more agents of hydride siloxane and vinyl siloxane. In some embodiments, the base also includes one or more of trisiloxane, disiloxane, dimethicone, and dimethiconol. In some embodiments, the base component and/or the activating component does not include a hydride functionalized polysiloxane.

One or both of the base component and the activating component may include one or more agents selected from the group consisting of reinforcing agent, feel modifiers, spreadability enhancers, adhesion modifiers, diluents, tack modifiers, optics modifiers, particles, volatile siloxanes, emulsifiers, emollients, surfactants, thickeners, solvents, film formers, humectants, preservatives and pigments. A cosmetic agent and/or a therapeutic agent can also be included in the base or the activating component.

Another aspect of the invention provides a method of improving skin aesthetics, including applying the base component followed by the activating component. A related aspect provides a film formed via this method.

### BRIEF DESCRIPTIONS OF THE DRAWINGS

FIG. 1 illustrates the pristine graphene physical chemical structure (an atomic-scale hexagonal lattice made of carbon atoms).

FIG. 2 illustrates the comparison of a sample product (formula #F100-245-37-7) of the present invention with a conventional product in terms of film edge peeling and breaking points.

FIG. 3 illustrates the comparison of a sample product (formula #F100-245-37-7) of the present invention with a conventional product in terms of film tear and tensile strength at breaking points.

FIG. 4 illustrates the response from 30 test participants on eye bag reduction (formula #F100-245-37-7).

FIG. 5 illustrates the response from 30 test participants on eye dark circle reduction (formula #F100-245-37-7).

FIG. 6 illustrates the response from 30 test participants on allergy reduction (formula #F100-245-37-7).

FIG. 7 illustrates the response from 30 test participants on product mechanic issue (formula #F100-245-37-7).

FIG. 8 illustrates the response from 30 test participants on water bearing issue (formula #F100-245-37-7).

FIG. 9 illustrates the response from 30 test participants on makeup application (formula #F100-245-37-7).

FIG. 10 illustrates the response from 30 test participants on reduction of wrinkles & fine lines around eyes (formula #F100-245-37-7).

FIG. 11 illustrates the response from 30 test participants on hydration issue (formula #F100-245-37-7).

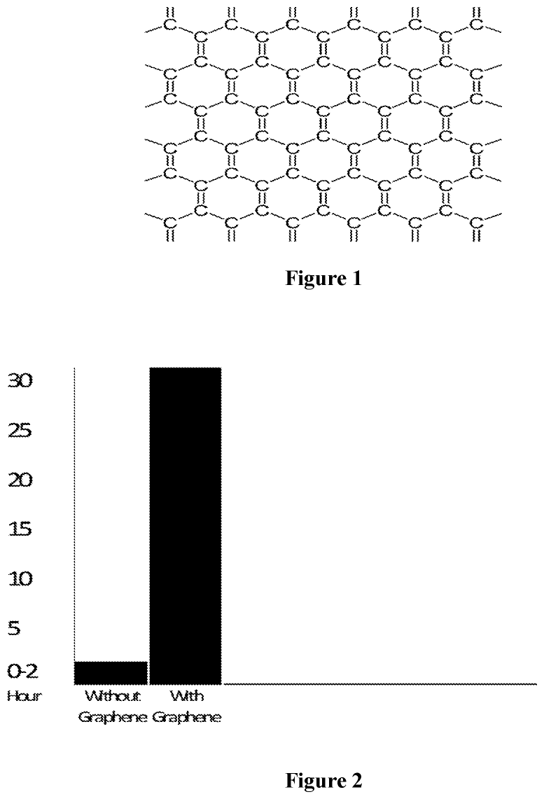

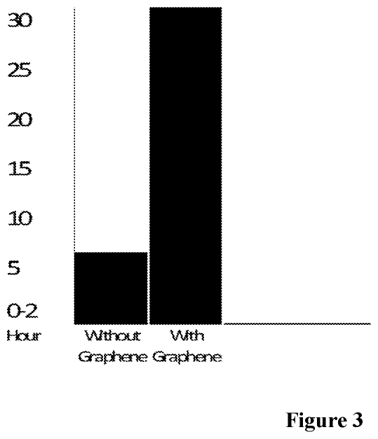

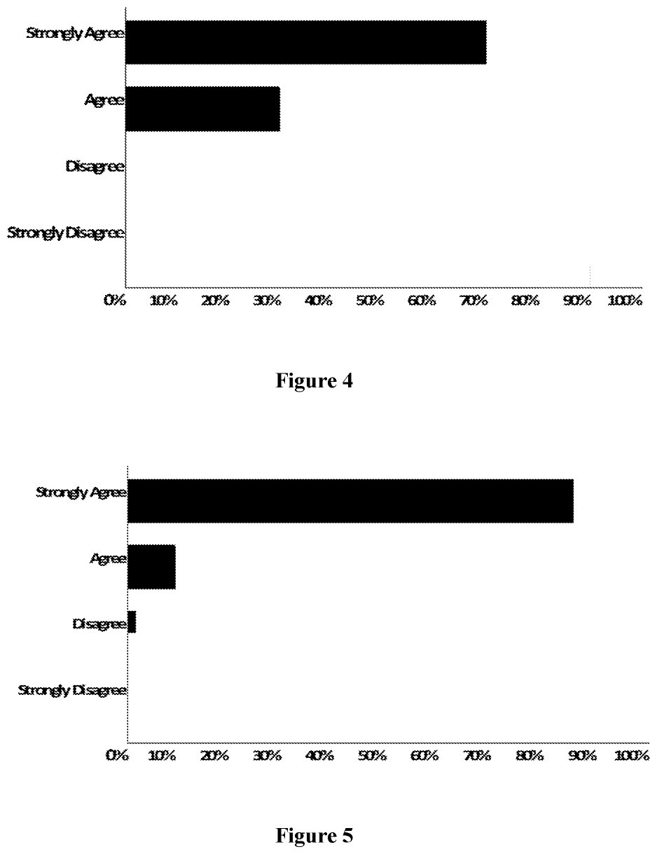

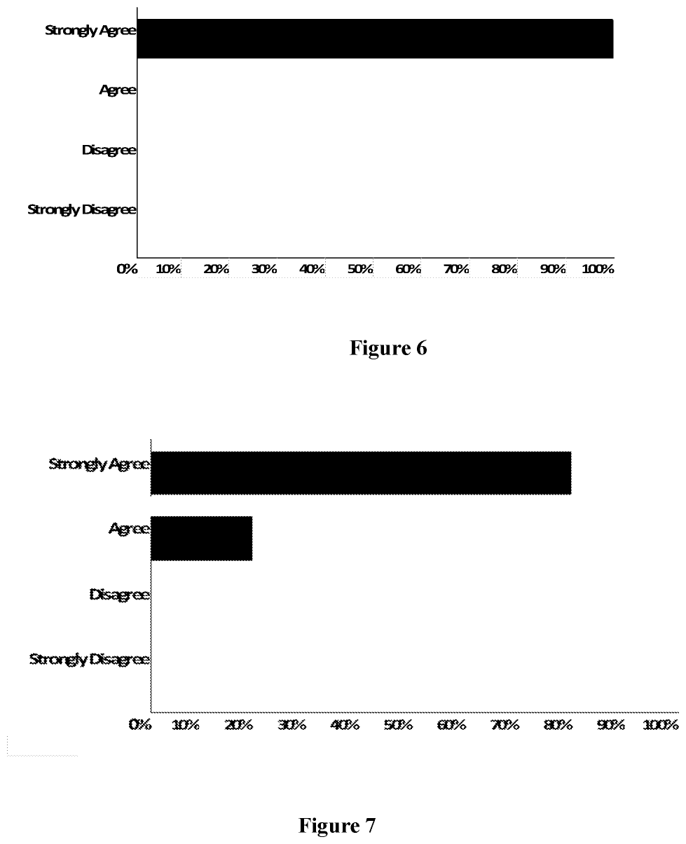

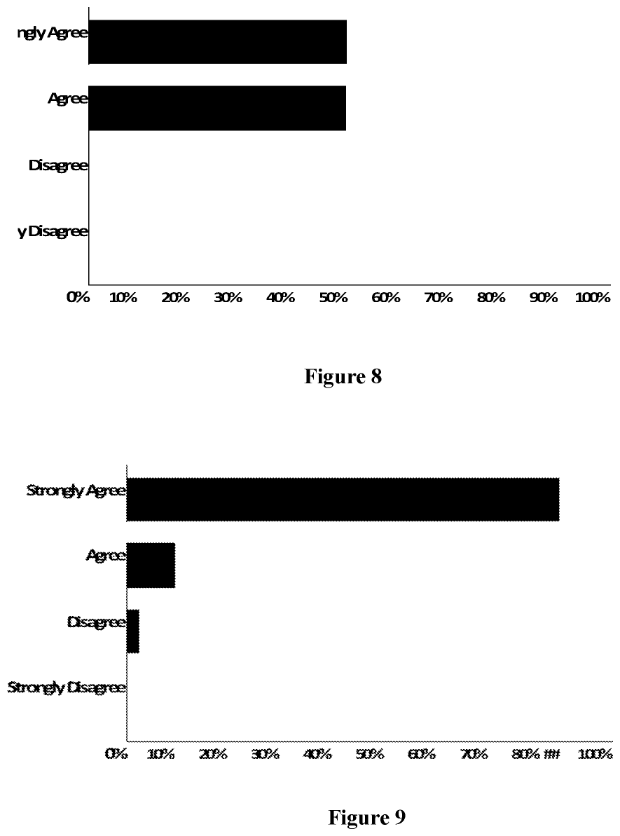

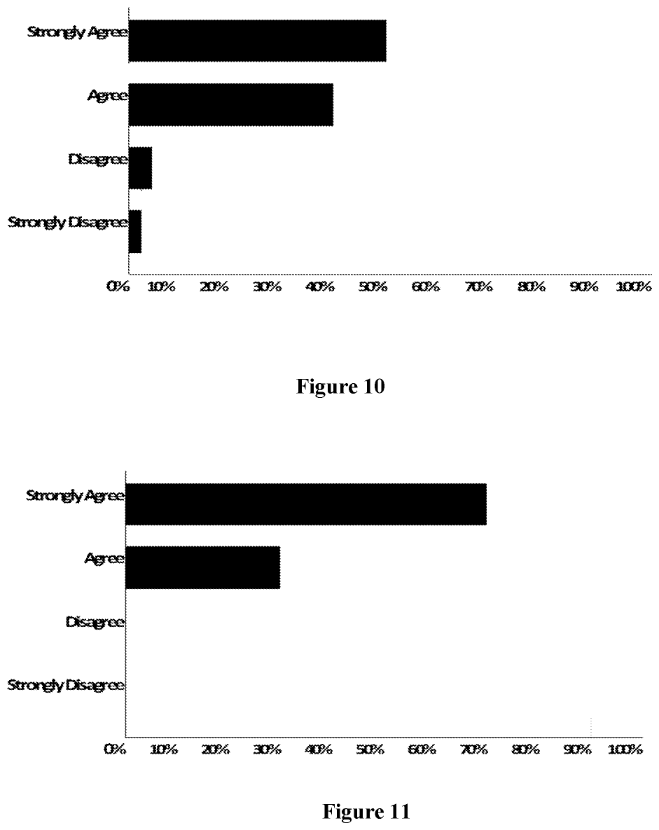

### DETAILED DESCRIPTION

This current invention discloses the compositions that can form an instant film after the application of 2-step cream and serum onto the skin. This film can mimic “young-looking skin” for cosmetics, make-up, or even for therapeutic and aesthetic application purpose. The main usage is for cosmetic applications for those who have aging-related appearance, e.g. skin blemishes, eye bags, wrinkles, enlarged pores, and age spot/brown spots, acne scars, birth and stretch marks. This current invention further discloses the medical grade of graphene and the natural Pressure Sensitive Adhesive (PSA) method to ensure this “young-looking skin” film has the strong properties to wear and cling onto the real skin for a much longer period of time than previous products, e.g. for 24 hours or longer. In the meantime, the wearer can perform and maintain normal daily activities without the same restrictions as prior products, without the risk of the physical film edges peeling or breaking, which will fail the whole functional performance. Therefore, the disclosed compositions of the invention have shown dramatic improvement of the viability and wearability of this film that mimics “young-looking skin” to truly benefit our aging and skin imperfection needs. It is a perfect problem-solving product, non-invasive, and safe to use.

While the following text may reference or exemplify specific embodiments of a modified polymer or a method of modifying the polymer, it is not intended to limit the scope of the invention to such particular reference or examples. Various modifications may be made by those skilled in the art, in view of practical and economic considerations, such as the amount of the hydrophilic group in the polymeric units and its ratio relative to the overall units of the polymer.

The articles “a” and “an” as used herein refers to “one or more” or “at least one,” unless otherwise indicated. That is, reference to any element or component of the present invention by the indefinite article “a” or “an” does not exclude the possibility that more than one element or component is present.

The term “about” as used herein refers to the referenced numeric indication plus or minus 10% of that referenced numeric indication.

An aspect of the document provides a kit for improving skin aesthetics. The kit generally includes (a) a base component, which contains graphene and a cross-linkable elastomer; and (b) an activating component containing an agent promoting the cross-linking of the elastomer.

The graphene is preferably in a form suitable for being mixed in a cosmetic formulation. In some embodiments, the graphene is processed via microwaving, vacuuming, ultrasonicating, or centrifuging before being introduced to component (a). The amount of the graphene may range from about 0.01% to about 30% by weight in the base component. In some embodiments, the graphene ranges from about 0.02% to about 25%, from about 0.02% to about 20%, from about 0.04% to about 20%, from about 0.02% to about 15%, from about 0.04% to about 15%, or from about 0.04% to about 10% by weight in the base component. In some embodiments, the amount of the graphene in the base component is about 100 ppm, about 200 ppm, about 500 ppm, about 800 ppm, about 1000 ppm or about 1500 ppm. In some embodiments, the graphene is a single layer. In some embodiments, the graphene is of multiple layers.

In some embodiments, component (a) is substantially free from water. In some embodiments, the water content in component (a) is less than about 5%, less than about 2%, less than about 1%, less than about 0.5%, less than about 0.2%, less than about 0.1% by weight.

The cross-linkable elastomer in the base component generally ranges from about 0.1% to about 25% by weight. In some embodiments, the cross-linkable elastomer in the base component ranges from about 0.1% to about 20%, from about 0.5% to about 20%, from about 0.5% to about 15%, or from about 1% to about 10% by weight.

In some embodiments, the cross-linkable elastomer is siloxane having a functional group for cross-linking. Non-limiting examples of such siloxane includes vinyl vinyl terminated polydimethylsiloxane; vinyl terminated diphenyl siloxane-dimethylsiloxane copolymers; vinyl terminated polyphenylmethylsiloxane, vinylphenylmethyl terminated vinyl-phenylsiloxane-phenylmethylsiloxane copolymer; vinyl terminated trifluoropropylmethyl-siloxane-dimethylsiloxane copolymer; vinyl terminated diethyl siloxane-dimethylsiloxane copolymer; vinylmethylsiloxane-dimethylsiloxane copolymers, vinyl terminated; monovinyl terminated polydimethylsiloxanes; and combinations thereof.

Additional exemplary elastomers include Cyclomethicone and Dimethicone Crosspolymer; Dimethicone and Dimethicone Crosspolymer; Cyclopentasiloxane and PEG-12 Dimethicone Crosspolymer; Cyclopentasiloxane and Dimethicone; Crosspolymer (and) Dimethicone/Vinyl Dimethicone; Crosspolymer (and) Dimethiconol. Any of these elastomers can be combined with one or more of the others.

The base component may further include a polysiloxane terminated with a hydroxyl group. In some embodiments, the hydroxyl-terminated polysiloxane is a poly-dimethyl siloxane.

The base component and/or the activating component can further include a pressure sensitive adhesive. The base component can be of translucent color or multiple tinted color. The base cream has a normal cosmetics cream viscosity. The rheology of the cream is easy to spread, and apply onto the skin. In some embodiments, the viscosity for the base component is from about 35,000 cps to about 280,000 cps at room temperature. In some embodiments, the viscosity for the activating component is from about 50 to about 50,000 cps at room temperature. In some embodiments, the room temperature is about 70° F.

In some embodiments, the activating component the agent for promoting cross-linking is a metal catalyst. Non-limiting examples of the metal catalyst includes platinum carbonyl cyclovinylmethylsiloxane complexes, platinum divinyltetramethyldisiloxane complexes, platinum cyclovinylmethylsiloxane complexes, platinum octanaldehyde/octanol complexes, Tris (dibutylsulfide) Rhodium trichloride, tin II octoate, Tin II neodecanoate, dibutyltin diisooctylmaleate, Di-n-butylbis(2,4 pentanedionate)tin, di-n-butylbutoxychlorotin, dibutyltin dilaurate, dimethyltin dineodecanoate, dimethylhydroxy(oleate)tin and tin II oleate, or a combination thereof.

The amount of the catalyst in the activating component generally ranges from about 0.05% to about 20% by weight. In some embodiments, the amount of the catalyst ranges from about 0.1% to about 20%, from about 0.5% to about 20%, from about 0.5% to about 15%, or from about 0.5% to about 15%.

The activating component can further include one or more siloxanes. None limiting examples include trisiloxane, disiloxane, dimethicone, and dimethiconol.

One or both of the base component and the activating component can further include one or more agents selected from feel modifiers, spreadability enhancers, adhesion modifiers, diluents, tack modifiers, optics modifiers, particles, volatile siloxanes, emulsifiers, emollients, surfactants, thickeners, solvents, film formers, humectants, preservatives and pigments.

In some embodiments, one or both of the base component and the activating component can further include a reinforcing agent selected from mica, zinc oxide, titanium dioxide, magnesium aluminum silicate, aluminum oxide, clay, silica and fumed silica. In some embodiments, one or both of the base component and the activating component can include at least one of a cosmetic agent and a therapeutic agent. For example, active anti-aging ingredients and traditional herbal medicines for long term anti-aging treatment benefits can be introduced into the base and/or activating component. These anti-aging active ingredients and traditional herbal medicines are designed to work underneath the film formed components for more effective and long term anti-aging benefits.

Another aspect of the invention provides method of improving skin aesthetics. The method generally includes applying to the skin the base component followed by applying to the skin the activating component. The base component and the activating component are as described above. The film thus formed serves for purposes such as cosmetics, make-up, or even for therapeutic and aesthetic application purpose.

Another related aspect of the invention provides a film for skin aesthetics, which includes graphene and cross-linked elastomer. The graphene ranges from about 0.01% to about 30% by weight in the film. The graphene in the film is a single layer or multiple layers.

Another aspect provides a method of manufacturing the base component described above. The method generally includes mixing graphene with a cross-linkable elastomer, wherein the graphene ranges from about 0.01% to about 30% by weight in the base component. The graphene can be processed before mixing via microwaving, vacuuming, ultrasonicating, and centrifuging. The graphene becomes completely dispersible, easier and more evenly to attach to the other raw materials after the above processing and can consistently form hexagonal lattice for more stronger and viable finished product. Specifically, the above processing improves one or more aspects of the graphene including purity, solubility, thickness, structure, surface morphology, pore volume, density, flake size. In some embodiments, the flake size ranges from about 0.5-20 microns. In some embodiments, the thickness ranges from about 0.5 to 10 nm. In some embodiments, the purity ranges from about 95%-100%.

In non-limiting examples, one of more of the following agents can be introduced to one or both of the base component and the activating component:

| | |
|---|---|
| Dow Corning ® 8500 Conditioning Agent | Bis (C13-15 Alkoxy) PG Amodimethicone |
| Astragulas Membranaceus Root Extract | Astragulas Membranaceus Root Extract |
| Dow Corning ® 9011 Silicone Elastomer Blend | Cyclopentasiloxane (and) PEG-12 Dimethicone |
| | Crosspolymer |
| Glycyrrhiza Uralensis (Licorice) Extract | Glycyrrhiza Uralensis (Licorice) Extract |
| Dow Corning ® 9040 Silicone Elastomer Blend | Cyclopentasiloxane (and) Dimethicone Crosspolymer |
| Angelica Polymorpha Sinensis Root Extract | Angelica Polymorpha Sinensis Root Extract |
| Dow Corning ® 9041 Silicone Elastomer Blend | Dimethicone (and) Dimethicone Crosspolymer |
| Zizyphus Jujuba Fruit Extract | Zizyphus Jujuba Fruit Extract |
| Dow Corning ® 9045 Silicone Elastomer Blend | Cyclopentasiloxane (and) Dimethicone Crosspolymer |
| White Poria Extract | White Poria Extract |
| Dow Corning ® 9046 Silicone Elastomer Blend | Dimethicone (and) Dimethicone Crosspolymer |
| Camellia Sinensis (Green Tea) Leaf Extract | Camellia Sinensis (Green Tea) Leaf Extract |
| Dow Corning ® 9509 Silicone Elastomer Suspension | Vinyl Dimethicone Crosspolymer (and) C12-14 Pareth-12 |
| Morus Alba (Mulberry) Leaf Extract | Morus Alba (Mulberry) Leaf Extract |
| Dow Corning ® 9546 Silicone Elastomer Blend | Cyclopentasiloxane (and) Dimethicone |
| | Crosspolymer (and) Dimethicone/Vinyl |
| | Dimethicone Crosspolymer (and) Dimethiconol |
| Coix Lacryma-Jobima-Yuen Seed Extract | Coix Lacryma-Jobima-Yuen Seed Extract |
| Panax Ginseng Root Extract | Panax Ginseng Root Extract |
| Dow Corning ® 9701 Cosmetic Powder | Dimethicone/Vinyl Dimethicone Crosspolymer (and) Silica |
| Silk Peptides | Silk Peptides |
| Dow Corning ® AMS-C30 Cosmetic Wax | C30-45 Alkyl Methicone (and) C30-45 Olefin |
| Chiralyst P407 | Bis(1,5-cyclooctadiene)rhodium(I) tetrafluoroborate |
| Silk Amino Acids | Silk Amino Acids |
| Dow Corning ® Toray FZ-2222 | Bis-Isobutyl PEG-24/PPG-7/Dimethicone |
| 1,3 Butylene Glycol | Butylene Glycol |
| “Dow Corning ® AP-8087 Fluid” | “Bis-Hydroxy/Methoxy Amodimethicone” |
| Glycerine 99.7% Natural Kosher | Glycerine 99.7% Natural Kosher |
| Dow Corning ® BY 11-030 | Cyclopentasiloxane (and) PEG/PPG-19/19 Dimethicone |
| Kaolin Clay | Kaolin |
| Dow Corning ® BY 25-337 | PEG/PPG-19/19 Dimethicone (and) C13-16 |
| | Isoparaffin (and) C10-13 Isoparaffin |
| Catalyst: 0.5-3.5%, 20% | Platinum Catalyst |
| Dow Corning TI-3011 | Dimethicone & Dimethiconol |
| Mica S-25 | Mica |
| Dow Corning ® 2501 Cosmetics Wax | Bis-PEG-18 Methyl Ether Dimethyl Silane |
| Glycolic Acid | Glycolic Acid |
| Dow Corning ® 2502 Cosmetics Fluid | Cetyl Dimethicone |
| Platinum C1142AF | Platinum Catalyst |
| Dow Corning ® 593 fluid | Dimethicone and Trimethylsiloxysilicate |
| Plantacare ® 818 UP | COCO-GLUCOSIDE |
| Dow Corning ® 670 fluid | Cyclopentasiloxane (and) Polypropylsilsesquioxane |
| Solubilisant LRI | PPG-26-Buteth-26 (and) PEG-40 Hydrogenated |
| | Castor Oil (and) Water |
| Dow Corning ® 680 ID fluid | Polypropylsilsesquioxane (and) Isododecane |
| Propylene Glycol | Propylene Glycol |
| Dow Corning ® 7-3099 Dimethicone HIP Emulsion | Dimethicone (and) Ceteth-10 (and) Laureth-4 |
| Dow Corning ® 7-3100 Gum Blend Hip Emulsion | Cyclopentasiloxane (and) Dimethiconol (and) |
| | Laureth-4 (and) Laureth-23 |
| VQM 199-200 | Silica Dimethylsilyloxy |
| Dow Corning ® 7-3101 Elastomer Blend Hip Emulsion | Cyclopentasiloxane (and) Dimethicone |
| | Crosspolymer (and) Dimethicone |
| Umicore CX11 | Bis(1,4-naphthoquinone)bis[1,3-bis(2,6 |
| | diisopropylphenyl)imidazol-2- |
| | ylidene]dipalladium(0) |
| PT-5017F | Platinum Catalyst |
| Dow Corning ® 7-3110 Volatile Fluid Hip Emulsion | Dimethicone (and) Trisiloxane (and) Ceteth-10 (and) |
| | Laureth-4 |
| Euxyl PE 9010 | Phenoxyethanol (and) Ethylhexylglycerin |
| Dow Corning ® 7-3120 VFAP Hip Emulsion | Dimethicone (and) Trisiloxane (and) Ceteth-10 (and) |
| | Laureth-4 (and) Aqua |
| Matrixyl 3000 | Glycerin (and) Aqua (and) Butylene Glycol (and) |
| | Carbomer (and) Polysorbate 20 (and) Palmitoyl |
| | Tripeptide-1 (and) Palmitoyl Tetrapeptide-7 |
| Dow Corning ® BY 25-339 Cosmetic Fluid | PEG/PPG-30/10 Dimethicone (and) Dipropylene Glycol |
| Chitosan | Chitosan |
| Dow Corning ® CE 8401 Emulsion | Bis-Isobutyl PEG/PPG-20/35/Amodimethicone |
| | Copolymer (and) Cetyl Ethylhexanoate (and) |
| | Polysorbate 80 (and) Butylene Glycol. |
| Dow Corning ® CE-1689 Smoothing Emulsion | Dimethicone (and) Amodimethicone (and) |
| | Laureth-23 (and) Polyquaternium-10 (and) Laureth-4 |
| Dow Corning ® Ce-1874 Microemulsion | PEG-7 Dimethicone (and) Laureth-7 (and) |
| | Polysorbate 20 |
| Dow Corning ® EL-7040 Hydro Elastomer Blend | Caprylyl Methicone (and) PEG-12 |
| | Dimethicone/PPG-20 Crosspolymer |
| CELL-U-LASH ™ 90 | Cellulose |
| Dow Corning ® EL-8040 Id Silicone Organic Blend | Isododecane (and) Dimethicone Crosspolymer |
| Jade Powder | Nephrite Extract |
| Dow Corning ® EL-8050 Id Silicone Org.Elast.blend | Isododecane (and) Dimethicone/Bis- |
| | Isobutyl PPG-20 Crosspolymer |
| Gemtone ® Garnet | Mica (and) Titanium Dioxide (and) Carmine (and) |
| | Iron Oxides |
| Dow Corning ® EL-9140 Dm Silicone Elastomer Blend | Dimethicone (and) Dimethicone Crosspolymer |
| Seamollient | ALGAE EXTRACT |
| Dow Corning ® EL-9240 Silicone Elastomer Blend | Dimethicone (and) Dimethicone Crosspolymer |
| Bronidox ® 1160 | Phenoxyethanol |
| Umicore M22 | cis-[1,3-Bis(2,4,6-trimethylphenyl)-2- |
| | imidazolidinylidene]dichloro(3-phenyl-1H-inden-1- |
| | ylidene)(triisopropylphosphite)ruthenium(II) |
| Dow Corning ® EP-9215 Cosmetic Powder | Dimethicone/Vinyl Dimethicone Crosspolymer |
| Plantacare ® 810 UP | CAPRYLYL/CAPRYL GLUCOSIDE |
| Dow Corning ® Rm 2051 Thickening Agent | Sodium Polyacrylate (and) Dimethicone (and) |
| | Cyclopentasiloxane (and) Trideceth-6 (and) |
| | PEG/PPG-18/18 Dimethicone |
| Dow Corning ® Vm-2270 Aerogel Fine Particles | Silica Silylate |
| Dow Corning ® 1515 Gum | DIMETHICONOL |
| SMS-1 SERICITE S+++ | Mica (And) Glycolipids |
| Dow Corning ® 3901 Liquid Satin Blend | Dimethicone (and)Dimethicone/Vinyl Dimethicone |
| | Crosspolymer |
| KoboGel ™ D5 Medium | Cyclopentasiloxane (And) Titanium Dioxide (And) |
| | Cyclohexasiloxane (And) Iron Oxides (CI 77492) |
| | (And) Iron Oxides (CI 77491) (And) PEG/PPG-18/18 |
| | Dimethicone (And) Iron Oxides (CI 77499) (And) |
| | Dimethicone/Vinyl Dimethicone Crosspolymer |
| | (And) Disteardimonium Hectorite (And) Tocopheryl |
| | Acetate (And) Isopropyl Titanium Triisostearate |
| | (And) Triethoxysilylethyl |
| Dow Corning MG7-9900A | Dimethicone and Dimethiconol Crosspolymer |
| Dow Corning MG7-9900B | Dimethicone and Dimethiconol Crosspolymer |
| SILIGHT DS-PDL3 | Silica (And) Dimethicone (And) Cetearyl Alcohol |
| | (And) Stearic Acid (And) Ceramide NP (And) |
| | Phytosterols |
| Dow Corning ® 9506 Powder | Dimethicone/Vinyl Dimethicone Crosspolymer |
| SP-10-FS | Nylon-12 (And) Periluorooctyl Tri ethoxysilane |
| Dow Corning ® 9591 Cosmetic Powder Dispersion | CYCLOPENTASILOXANE POLYSILICONE-11 |
| NYLON 10-12 | Nylon-12 (And) Isopropyl Titanium Triisostearate |
| Dow Corning ® El-9241 Dm Silicone Elastomer Blend | DIMETHICONE CROSSPOLYMER |
| CELLULOBEADS D-50 | Cellulose |
| Dow Corning ® Fz-2233 | Bis-Isobutyl PEG/PPG-10/7/Dimethicone Copolymer |
| MSS-500 | Silica |
| Dow Corning ® Pf-9510 Elastomer | Dimethicone/Vinyl Dimethicone. Crosspolymer and |
| | C12-14 Pareth-12 |
| KTZ ® DIAMOND BRIGHT | Mica (And) Titanium Dioxide |
| Dow Corning ® El-9048 Silicone Elastomer Blend | Cyclopentasiloxane (and) Dimethicone Crosspolymer |
| KTZ ® SUPERB SILVER | Mica (And) Titanium Dioxide |
| MTM3K50XZ4 | Zinc Oxide (And) Methyl Trimethicone (And) PEG-10 |
| | Dimethicone (And) Methicone |
| Xiameter ® Pmx-0244 | Cyclotetrasiloxane |
| BPD-500W | HDI/Trimethylol Hexyllactone Crosspolymer (And) |
| | Silica |
| Pro-Structure | Hydrolyzed Walnut Extract |
| HBP45M160 | Butyloctyl Salicylate (And) Titanium Dioxide (And) |
| | Alumina |
| | (And) Stearic Acid (And) Polyhydroxystearic Acid |
| Xiameter ® Pmx-0245 | Cyclopentasiloxane |
| CMF655T4* | Titanium Dioxide (And) Cyclopentasiloxane (And) |
| | Polyglyceryl-3 Polydimethylsiloxyethyl |
| | Dimethicone (And) Alumina (And) Hydrogen |
| | Dimethicone |
| DL-PANTHENOL | DL-PANTHENOL |
| Xiameter ® Pmx-0246 | Cyclohexasiloxane (and) Cyclopentasiloxane |
| CM3K40T4* | Cyclopentasiloxane (And) Titanium Dioxide (And) |
| | PEG-10 Dimethicone (And) Alumina (And) |
| | Hydrogen Dimethicone |
| Xiameter ® Pmx-0344 | Cyclotetrasiloxane (and) Cyclopentasiloxane |
| EcoSense ™ 919 | Coco-Glucoside |
| Gcc 300kc | Caprylic/Capric Triglyceride |
| ESCULANE ™ | Caprylic/Capric Triglyceride - Algae Extract - |
| | Laminaria Digitata Extract |
| Givobio GZN | Zinc Gluconate |
| Xiameter ® Pmx-0345 | Cyclopentasiloxane (and) Cyclohexasiloxane |
| GELAID ® SILK | Cyclohexasiloxane (and) Vinyl |
| | Dimethicone/Methicone Silsesquioxane (and) |
| | Acrylamide/Sodium Acryloyldimethyltaurate |
| | Copolymer (and) Isohexadecane (and) Polysorbate |
| | 80 (and) PEG -10 Dimethicone |
| Hydrolite 5 | Pentylene Glycol |
| Xiameter ® Pmx-1403 Fluid | Dimethicone (and) Dimethiconol |
| TR-1 | Nylon-6 |
| SEPILIFT ™ DPHP | Dipalmitoyl hydroxyproline |
| L-ASCORBIC ACID USP | ASCORBIC ACID |
| SunSpheres ™ Powder | Acrylates Copolymer |
| Xiameter ® Pmx-1411 Fluid | Cyclopentasiloxane (and) Dimethicone |
| CELLOSIZE ™ PCG-10 | Hydroxyethylcellulose |
| Crotein ™ A | Hydrolyzed Collagen |
| Xiameter ® Pmx-1501 Fluid | Cyclopentasiloxane (and) Dimethiconol |
| EcoSmooth ™ Satin Conditioning Polymer | Ethylene/Sodium Acrylate Copolymer |
| Xiameter ® Pmx-1503 Fluid | Dimethicone (and) Dimethiconol. |
| ACULYN ™ 88 Rheology Modifier | Acrylates/Steareth-20 Methacrylate Crosspolymer |
| Xiameter ® Pmx-1556 Fluid | Phenyl Trimethicone (and) Dimethiconol |
| ACULYN ™ 38 Rheology Modifier | Acrylates/Vinyl Neodecanoate Crosspolymer |
| SP-10 | Nylon-12 |
| Magnesium Aluminum Silicate | Magnesium Aluminum Silicate |
| Xiameter ® 1184 | Dimethicone (and) Trisiloxane |
| DIMETHISIL ® DM-5~l,000,000 | Dimethicone |
| Xiameter ® Pmx-200 Silicone Fluid | Dimethicone |
| VOLASIL ™ 8515 | Cyclotetrasiloxane (and) Cyclopentasiloxane |
| Andisil ® SF 0.65, 1, 1.5, 2~5,000 | Disiloxane, Trisiloxane, Dimethicone |
| ELIGHT SDS | Dimethicone/Vinyl Dimethicone Crosspolymer |
| | (And) Silica (And) Dimethicone |
| CHEMSIL K-51 | Cyclopentasiloxane (and) Dimethicone/Vinyl |
| | Dimethicone Crosspolymer |
| Andisil ® VS 200, 500, 1,000, 5000 | Vinyl Dimethicone |
| KSP-300 | Diphenyl Dimethicone/Vinyl Diphenyl Dimethicone/ |
| | Silsesquioxane Crosspolymer |
| AEROSIL ® R 106 | Cryclotetrasiloxane, octametryl-, reaction products |
| | with silica |
| VITAMIN E USP | DL-ALPHA TOCOPHERYL ACETATE |
| Andisil ® XL-1B, 10, 11, 12, 13, 17 | Si—H functional |
| AEROSIL ® R 8200 | Silanamine, 1,1,1,-trimethyl-N-(trimetrysilyl)-, |
| | hydrolysis products with silica |
| BOTANAGEN | 3-AMINOPROPYL DIHYDROGEN PHOSPHATE |
| Andisil ® CE-4, 13, 500 Chain Extender | terminal Si—H functional |
| KSG-15 | Cyclopentasiloxane (and) Dimethicone/Vinyl |
| | Dimethicone Crosspolymer |
| Silbione RTV 4420A | Dimethcone and Dimethiconol |
| SilCare Silicone 15M50 | PHENYL TRIMETHICONE |
| SILICA SHELLS | Silica |
| JEESILC PS-DMBF | DIMETHICONE, BIS VINYL |
| | DIMETHICONE/DIMETHICONE COPOLYMER |
| Silbione RTV 4420B | Caprylyl Methicone |
| MAP SL | MAGNESIUM ASCORBYL PHOSPHATE |
| JEESILC PDS-1/1.5/5/20/200/350/500/1000 | Dimethicone |
| Aristoflex AVC | Ammonium Acryloyldimethyltaurate/VP Copolymer |
| Cabosil TS-720 | Fumed Silica |
| Milk Tein NPNF ® | Hydrolyzed Milk Protein |
| JEECOL CS-12 | CETEARETH-12 |
| SK-INFLUX ® V | CARBOMER, CERAMIDE 1, CERAMIDE 3, |
| | CERAMIDE 6 II CHOLESTEROL, |
| | PHYTOSPHINGOSINE, SODIUM LAUROYL |
| | LACTYLATE, XANTHAN GUM |
| Cabosil TS-610 | Fumed Silica |
| JEECHEM OP-12 | OCTOXYNOL-12 |
| KF-995 | CYCLOPENTASILOXANE |
| Cabosil TS-530 | Fumed Silica |
| Carbopol ® Ultrez 10, 20, 21, 30 polymer | Carbomer/Acrylates/C10-30 Alkyl Acrylate |
| | Crosspolymer |
| Cabosil TS-500 | Fumed Silica |
| CERAMIDE III, IIIB | Ceramide NP |
| JEESILC PS-CMBF | CYCLOPENTASILOXANE, BIS VINYL |
| | DIMETHICONE/DIMETHICONE COPOLYMER |
| Reolosil QS-10LS | Fumed Silica |
| SilCare Silicone 1M71 | Stearoxytrimethylsilane |
| Reolosil QS-20L | Fumed Silica |
| JEESILC PS-DMLV | DIMETHICONE, BIS VINYL |
| | DIMETHICONE/DIMETHICONE COPOLYMER |
| Aristoflex AVS | Sodium Acryloyldimethyltaurate/VP Crosspolymer |
| Orisil M130 | Fumed Silica |
| Aristoflex HMB | Ammonium Acryloyldimethyltaurate/Beheneth-25 |
| | Methacrylate Crosspolymer |
| Orisil M 200 | Fumed Silica |
| Carbopol ® 934 polymer. 940, 941, 980, | CARBOMER |
| Orisil M300 | Fumed Silica |
| Andisil VQM 0.6, 1, 6, 60, 100, 1040, 2050. 199-200 | Vinyl Resin Additive |
| SilCare Silicone 31M50 | Caprylyl Trimethicone |
| Zeosil 115GR | Silica |
| SK-INFLUX ® | Ceramide 3; Ceramide 6 II; Ceramide |
| | 1; Phytosphingosine; Cholesterol; Sodium Lauroyl |
| | Lactylate; Carbomer; Xanthan Gum.Conservanti: |
| | Methylparaben (max. 0.3%), Propylparaben (max. |
| | 0.2%). |
| Zeosil 125GR | Silica |
| JEECHEM FS 102 | HEXYLENE GLYCOL, PEG-25 |
| | HYDROGENATED CASTOR OIL, PEG-40 |
| | HYDROGENATED CASTOR OIL |
| AEROSIL ® R 812 S | SILICA SILYLATE |
| Zeosil 175GR | Silica |
| AEROSIL ® R 7200 | 2-Propenoic acid, 2-methyl-, 3- |
| | (trimethoxysilyl)propylester, reaction products with |
| | silica |
| Zeosil 185GR | Silica |
| KP-561P | ACRYLATES/STEARYL |
| | ACRYLATE/DIMETHICONE METHACRYLATE |
| | COPOLYMER |
| JEESORB O-20 NF | POLYSORBATE 80 |
| Zeosil 215GR | Silica |
| KP-545L | ACRYLATES/DIMETHICONE COPOLYMER |
| | DIMETHICONE |
| Zeosil Premium 200MP | Silica |
| JEECHEM NP-12 | NONOXYNOL-12 |
| KF-9028 | CYCLOPENTASILOXANE DIMETHICONE |
| JEESORB L-20 NF | POLYSORBATE 20 |
| Zeosil 1115MP | Silica |
| KF-7312J | CYCLOPENTASILOXANE |
| | TRIMETHYLSILOXYSILICATE |
| Zeosil 1165MP | Silica |
| KF-9021 | CYCLOPENTASILOXANE |
| TRIMETHYLSILOXYSILICATE | |
| Zeosil 142 | Silica |
| KF-96L, 96A, 96, 96H | DIMETHICONE |
| Zeosil 132 | Silica |
| Unisil NK3HS | Fumed Silica |
| Andiform C100, C106, C300, C1000, C1, 300 | Bis-Vinyldimethicone |
| SilCare Silicone 41M15 | Fumed Silica |
| AEROSIL ® R 812 | SILICA SILYLATE |
| JEECHEM NP-10 | NONOXYNOL-10 |
| KF-9008 | CYCLOPENTASILOXANE DIMETHICONE |
| Granpowder PSQ-Pt | Polymethylsilsesquioxane (and) Platinum |
| KF-9011 | CYCLOPENTASILOXANE DIMETHICONE |
| Gransurf 61 | PEG-12 Dimethicone |
| KF-9014 | CYCLOPENTASILOXANE DIMETHICONE |
| X-21-5495 | CYCLOPENTASILOXANE DIMETHICONE |
| X-21-5666 | CYCLOPENTASILOXANE DIMETHICONOL |
| KSG-15 | CYCLOPENTASILOXANE |
| | DIMETHICONE/VINYL DIMETHICONE |
| | CROSSPOLYMER |
| USG-103 | CYCLOPENTASILOXANE |
| | DIMETHICONE/VINYL DIMETHICONE |
| | CROSSPOLYMER |
| X-21-5250 | CYCLOPENTASILOXANE |
| | TRIMETHYLSILOXYSILICATE |
| TSPL-30-D5 | Cyclopentasiloxane (and) Norbornene/Tris |
| | (Trimethylsiloxy)Silylnorbornene Copolymer |
| Gransil GTS | Ethyl Trisiloxane (and) Polysilicone-11 |
| KSG-210 | Dimethicone (and) Dimethicone/PEG-10/15 |
| | Crosspolymer |
| KSG-240 | Cyclopentasiloxane (and) Dimethicone/PEG-10/15 |
| | Crosspolymer |
| GI CD-965 | Dimethicone (and) Stearoxymethicone/Dimethicone |
| | Copolymer (and) Dimethicono |
| KSG-310 | Mineral Oil (and) PEG-15/Lauryl Dimethicone |
| | Crosspolymer |
| GI CD-965 | Dimethicone (and) Stearoxymethicone/Dimethicone |
| | Copolymer (and) Dimethicono |
| KSG-320 | Isododecane (and) PEG-15/Lauryl Dimethicone |
| | Crosspolymer |
| Gransil DM-10/20/6 | Dimethicone (and) Polysilicone-11 |
| KSG-330 | Triethylhexanoin (and) PEG-15/Lauryl Dimethicone |
| | Crosspolymer |
| Gransil DMAM | Dimethicone (and) Caprylyl Methicone (and) |
| | Polysilicone-11 |
| KSP-101 | VINYL DIMETHICONE/METHICONE |
| | SILSESQUIOXANE CROSSPOLYMER |
| Granpowder Silica | Silica |
| KSP-102 | VINYL DIMETHICONE/METHICONE |
| | SILSESQUIOXANE CROSSPOLYMER |
| Granactive Oligopeptide-10 | Cyclopentasiloxane (and) Dimethicone (and) Water |
| | (and) Polysilicone-11 (and) Butylene Glycol (and) |
| | Decyl Glucoside (and) Oligopeptide-10 |
| KSP-105 | VINYL DIMETHICONE/METHICONE |
| | SILSESQUIOXANE CROSSPOLYMER |
| Gransurf 67 | PEG-10 Dimethicone |
| KSP-411 | POLYSILICONE-1 CROSSPOLYMER |
| CARESIL ™- XG CXG - 1101 | CYCLOPENTASILOXANE |
| | DIMETHICONE/VINYL DIMETHICONE |
| | CROSSPOLYMER |
| KSP-441 | POLYSILICONE-22 |
| Granacrysil BMAS | Isododecane (and) Isobutylmethacrylate/Bis- |
| | Hydroxypropyl Dimethicone Acrylate Copolymer |
| KF-9901 | Hydrogen Dimethicone |
| BELSIL ® PDM 1000 | Trimethylsiloxyphenyl Dimethicone |
| KF-9908 | TRIETHOXYSILYLETHYL |
| | POLYDIMETHYLSILOXYETHYL |
| | DIMETHICONE |
| BELSIL ® DM 1 PLUS, DM 5/10/20/50/100/500/1000 | DIMETHICONE |
| KF-9909 | TRIETHOXYSILYLETHYL |
| | POLYDIMETHYLSILOXYETHYL HEXYL |
| | DIMETHICONE |
| CARESIL ™- XG CXG - 1103 | CYCLOHEXASILOXANE |
| | CYCLOPENTASILOXANE DIMETHICONE/ |
| | VINYL DIMETHICONE CROSSPOLYMER |
| CARESIL ™- CSG-1001 | Aqua (and) Dimethicone (and) Dimethicone/Vinyl |
| | Dimethicone Crosspolymer (and) Dimethiconol |
| | (and) Butylene Glycol (and) Carbomer(and) |
| | Phenoxyethanol (and) Sodium Hydroxide |
| BELSIL ® REG 1102 | Dimethicone (and) |
| | Dimethicone/Vinyltrimethylsiloxysilicate |
| | Crosspolymer |
| CARESIL ™- F CSF - 3504 | Dimethicone (and) Dimethiconol |
| CARESIL ™- PF CPF - 3300 | Phenyl Trimethicone |
| Mirasil Micropearl 40 | Polymethylsilsesquioxane |
| CARESIL ™- FF CFF - 3401 | Trifluoropropyl Dimethicone |
| BELSIL ® TMS 803 | Trimethylsiloxysilicate |
| CARESIL ™- V CSV - 3002 | Cyclohexasiloxane |
| BELSIL ® CM 1000 | CYCLOPENTASILOXANE DIMETHICONOL |
| BELSIL ® CM 3092 | CYCLOPENTASILOXANE DIMETHICONOL |
| BELSIL ® CM 740 | CYCLOPENTASILOXANE DIMETHICONOL |
| HDK ® H2000 | Silica Dimethyl Silylate HYDROXYPROPYL |
| | CYCLODEXTRIN |
| BELSIL ® DM 3096 | DIMETHICONE DIMETHICONOL |
| BELSIL ® EG 5 | Cyclopentasiloxane (and) Dimethicone/Vinyl |
| | Dimethicone Crosspolymer |
| HDK ® H15 | Silica |
| BELSIL ® RG 100 | CYCLOPENTASILOXANE DIMETHICONE/ |
| | VINYLTRIMETHYL SILOXYSILICATE |
| | CROSSPOLYMER |
| HDK ® H15P | Silica |
| Mirasil CM5 | CYCLOPENTASILOXANE |
| HDK ® V15 | Silica |
| COSMETIC FLUID 8100-OH | Trisiloxane (and) Dimethicone (and) Dimethiconol |
| HDK ® V15P | Silica |
| EMULSIL ® WO-5115 | Cyclopentasiloxane (and) PEG/PPG-18/18 |
| | Dimethicone |
| HDK ® N20 | Silica |
| Mirasil CM5 HP | CYCLOPENTASILOXANE |
| HDK ® N20P | Silica |
| Mirasil D-DML LV | Dimethicone (and) Dimethiconol |
| HDK ® T30 | Silica |
| COSMETIC FLUID EU | Cyclohexasiloxane (and) Dimethiconol |
| HDK ® T40 | Silica |
| Mirasil DM 60 000 | Dimethicone |
| HDK ® H20 | Silica |
| COSMETIC FLUID 1585-OH | Cyclopentasiloxane (and) Dimethiconol |
| HDK ® H30 | Silica |
| Mirasil C-DMX 12 | Cyclopentasiloxane (and) Dimethicone |
| | Crosspolymer |
| HDK ® H18 | Silica |
| COSMETIC FLUID 9109-OH | Cyclotetrasiloxane (and) Cyclopentasiloxane (and) |
| | Dimethiconol |
| HDK ® N20D | Silica |
| COSMETIC FLUID 8503-HALS | Cyclopentasiloxane (and) Dimethiconol (and) |
| | Propoxytetramethyl Piperidinyl Dimethicone |
| Mirasil C-DML MV | CYCLOPENTASILOXANE (and) |
| | DIMETHICONOL |
| EMULSIL ® WO-3115 | Cyclopentasiloxane(and) Cyclotetrasiloxane (and) |
| | PEG/PPG-18/18 Dimethicone |
| COSMETIC FLUID 6570-DM | Disiloxane (and) Cyclopentasiloxane (and) |
| | Dimethicone |
| Mirasil CM5 HP | CYCLOPENTASILOXANE |
| CHEMSIL K-27 | Cyclopentasiloxane (and) Dimethiconol (and) |
| | Dimethicone/Vinyl Dimethicone Crosspolymer |
| Mirasil PEG 12 DM | PEG-12 DIMETHICONE |
| VOLASIL ™ DM-1.5 | Dimethicone |
| Mirasil C-DMP | CYCLOPENTASILOXANE (and) PEG/PPG-18/18 |
| | DIMETHICONE |
| COSMETIC FLUID 4120-DM | Cyclopentasiloxane (and) Polysilicone-22 |
| CHEMSIL K-72 | Trimethylsiloxysilicate |
| COSMETIC FLUID 0515-OH | Dimethicone Dimethiconol |
| SHINEBLEND ® 9710 | Cyclopentasiloxane (and) Cyclotetrasiloxane (and) |
| | Dimethicone |
| COSMETIC FLUID 1406-DM/OH | Dimethicone |
| GELAID ® 8205 | Cyclopentasiloxane (and) Dimethicone |
| COSMETIC FLUID 1486-DM | Cyclopentasiloxane (and) Dimethicone |
| GELAID ® 8317 | Cyclopentasiloxane (and) Cyclotetrasiloxane (and) |
| | Dimethicone |
| COSMETIC FLUID 1486-OH | Cyclopentasiloxane (and) Cyclotetrasiloxane (and) |
| | Dimethiconol |
| EMULSIL ® S-393 | PEG-12 Dimethicone |
| COSMETIC FLUID 1684-DM | Cyclopentasiloxane (and) Cyclotetrasiloxane (and) |
| | Dimethicone |
| EMULSIL ® S-399 | Lauryl PEG-9 Polydimethylsiloxyethyl Dimethicone |
| COSMETIC FLUID 3569-DM | Cyclopentasiloxane (and) Dimethicone |
| CHEMSIL K-12 | Dimethicone (and) Dimethicone PEG-10/15 |
| | Crosspolymer |
| COSMETIC FLUID 6520-DM | Disiloxane (and) Dimethicone |
| VOLASIL ™ 990~996 | Cyclotetrasiloxane |
| COSMETIC FLUID 6590-DM | Disiloxane (and) Dimethicone |
| VOLASIL ™ 7525 | Cyclopentasiloxane (and) Cyclohexasiloxane |
| COSMETIC FLUID 7007-DM | Dimethicone (and) Dimethicone/Vinyl Dimethicone |
| | Crosspolymer |
| VOLASIL ™ DM-0.65 | Disiloxane |
| COSMETIC FLUID 8108-OH | Dimethicone (and) Dimethiconol (and) Trisiloxane |
| VOLASIL ™ 1585 | Cyclopentasiloxane(and) Cyclotetrasiloxane |
| COSMETIC FLUID 8110-DMC | Dimethicone (and) Methyl Trimethicone (and) |
| | Dimethiconol (and) Dimethicone/Vinyl Dimthicone |
| | Crosspolymer (and) Trisiloxane |
| COSMETIC FLUID 8302-DM | Cyclotetrasiloxane (and) Dimethicone |
| VOLASIL ™ DM-1 | Trisiloxane |
| COSMETIC FLUID 9005-DM | Cyclopentasiloxane(and) Cyclotetrasiloxane (and) |
| | Dimethicone |
| CHEMSIL K-13 | Cyclopentasiloxane (and) Dimethicone/Vinyl |
| | Dimethicone Crosspolymer |
| COSMETIC FLUID 9109-DM | Cyclotetrasiloxane (and) Cyclopentasiloxane (and) |
| | Dimethicone |
| CHEMSIL K-17 | Dimethicone (and) Dimethicone/Vinyl Dimethicone |
| | Crosspolymer |
| COSMETIC FLUID 9502-DM | Cyclopentasiloxane (and) Dimethicone/Vinyl |
| | Dimethicone Crosspolymer |
| CHEMSIL K-45 | Acrylate/Dimethicone Copolymer (and) |
| | Cyclopentasiloxane |
| VOLASIL ™ 8100 | Dimethicone (and) Trisiloxane |
| CHEMSIL K-61 | Dimethicone (and) Dimethicone/Vinyl Dimethicone |
| | Crosspolymer |
| CHEMSIL K-73 | Cyclopentasiloxane (and) Trimethylsiloxysilicate |
| CHEMSIL SPD | Dimethicone/Dimethicone Vinyl Crosspolymer |

### Examples

The following examples illustrate ingredients for base component and activating component. It should be understood that these examples are not to be considered a disclaimer of alternative embodiments of the kit of the present invention.

### Formulation:

### F100-208-28-1

### Dow Corning MG7-9900A

0.5-25%

### Xiameter® 345

0.25-12%

### Unisil NK3HS

0.8-15.5

### Xiameter 1184

2-10%

### Andsil XL-17

0.25-5.5%

### Andisil® CE500

0.75-12%

### Dow Corning 9509

1-8%
Graphene complex
0.05-8%

### Nephrite Extract

0.5-5%

### Titanium Dioxide

0.25-3%

### Zirconium Dioxide

0.15-1.2%

### Gemtone® Garnet

0.01-2.5%

### Aluminum Dioxide

0.15-5%

### Phenoxyethanol

0.5-1%

### Dow Corning MG7-9900B

0.5-25%

### Xiameter 0244

1-10%

### Xiameter® Pmx-1501 Fluid

1-12%

### Dow Corning® 9041

8-18%

### Plantacare 818UP

1-7%

### Phenoxyethanol

0.5-1.5%

### Glycerine 99.7%

1-12%

### Platinum C1142AF

0.25-15.5%

### 1,3 Butylene Glycol

0.5-17%

### Propylene Glycol

0.5-18%

### F100-212-85-2

### Silbione RTV 4420A

0.5-22%

### Zeosil Premium 200MP

0.5-15.5%

### Andiform C300

0.25-15.5%

### Dow Corning® Pf-9510 Elastomer

1-8%

### Andisil XL-13

0.25-11%

### Andisil SF 500

1-11.5%
Graphene complex
0.05-18%

### Andisil® CE 13

### VOLASIL™ DM-1.5

1-12.5%

### COSMETIC FLUID 6570-DM

0.5-8%
0.25-7.5%

### Titanium Dioxide

0.25-3%

### Zirconium Dioxide

0.15-1.2%

### Mica S-25

0.01-7%

### KTZ® SUPERB SILVER

0.05-2%

### Aluminum Dioxide

0.15-5%

### Phenoxyethanol

0.5-1%

### Silbione RTV 4420B

0.5-22%

### Dow Corning® 9509

0.5-12%

### Dow Corning TI-3011

0.25-6%

### Dow Corning® 9045

0.25-15%

### Xiameter 245

1-13%

### Nephrite Extract

0.5-5%

### Mirasil C-DMX 12

0.5-8%

### Dow Corning® 9011

1-15%

### CHEMSIL K-51

1-8%

### Astragulas Extract

0.5-15%

### Licorice Extract

0.5-12%

### Matrixyl 3000

0.5-8%

### Glycerine 99.7%

1-15%

### 1,3 Butylene Glycol

0.5-18%

### PT5017F

0.25-12%

### Propylene Glycol

2-15%

### F100-217-55-3

### Dow Corning® EL-9140

1-20%

### Andiform C1000

0.5-15%

### Andiform C106

0.5-12%

### Cabosil TS-500

1-18%

### Xiameter 0245

1-20%

### GI CD-10/11

1-25%

### Andisil XL-12

0.15-25%

### COSMETIC FLUID 8110-DMC

0.5-8%

### Kaolin Clay

0.5-17%

### Xiameter 200 5 cSt

1-15%
Graphene complex
0.05-21.5%

### Mica S-25

0.15-10%

### Titanium Dioxide

0.25-3%

### Zirconium Dioxide

0.15-1.2%

### KTZ® SUPERB SILVER

0.01-7.5%

### Aluminum Dioxide

0.15-5%

### Phenoxyethanol

0.5-1%

### Granacrysil BMAS

0.5-15%

### CHEMSIL K-12

1-25%

### Mirasil DM 60 000

1-12%

### Dow Corning® 9046

1-20%

### KTZ® DIAMOND BRIGHT

0.25-5%

### Aristoflex AVS

0.25-8%

### CM3K40T4*

0.3-3.5%

### KSP-105

0.5-7.5%

### HDK® H30

0.25-12%

### VOLASIL™ DM-1.5

1-10%

### COSMETIC FLUID 9109-OH

1-12%

### Licorice Extract

0.25-15%

### Jujube Extract

0.25-10%

### PT5017F

0.25-12%

### Astragulas Extract

0.25-8%

### 1,3 Butylene Glycol

0.25-18%

### Glycerine 99.7%

1-15%

### Propylene Glycol

1-18%

### Phenoxyethanol

0.25-2.5%

### Titanium Dioxide

0.25-3%

### Zirconium Dioxide

0.15-1.2%

### F100-218-28-4

### Dow Corning® EL-9240

0.25-22%

### KSG-210

1-28%

### Andiform C300

1-35%

### Andisil CE500

1-25%

### Xiameter® Pmx-1501 Fluid

1-18%

### Andisil XL-11

0.05-15%

### Xiameter 0345

1-25%

### Andisil SF1.00

0.5-10%
Graphene complex
0.05-8%

### DCTI-3011

0.25-12%

### Cabosil TS-720

0.15-15%

### Euxyl PE 9010

0.25-5%

### KoboGel™ D5 Medium

0.15-5.5%

### MSS-500

0.15-3%

### Titanium Dioxide

0.25-3%

### Zirconium Dioxide

0.15-1.2%

### Aluminum Dioxide

0.15-5%

### COSMETIC FLUID 7007-DM

1-12%

### Dow Corning® 9041

1-10%

### CHEMSIL K-61

1-8%

### KF-9908

1-12%

### Dow Corning® 2502 Cosmetics Fluid

1-15%

### CELL-U-LASH™ 90

0.25-5.5%

### TSPL-30-D5

0.15-3.5%

### SilCare Silicone 31M50

0.25-5%

### Granpowder PSQ-Pt

0.25-1%

### Glycolic Acid

0.05-5.5%

### Aristoflex HMB

0.15-3.5%

### Granactive Oligopeptide-10

1-12%

### Chrysanthemum Extract

1-10%

### Licorice Extract

1-8%

### Platinum Catalyst

0.25-12%

### Seamollient

1-8%

### GELAID SILK

1-12%

### Glycerine 99.7%

1-15%

### 1,3 Butylene Glycol

1-18%

### Propylene Glycol

1-17%

### Phenoxyethanol

0.25-3.5%

### F100-237-95-5

### HDK® H18

0.5-8%

### Andiform C300

0.5-7%

### Andiform C106

0.25-3%

### Andisil® SF 0.65

1-15%

### Xiameter® Pmx-0245

2-10%

### Zeosil 215GR

0.5-10%
Graphene complex
0.05-8.5%

### Dow Corning® 9506 Powder

0.25-7%

### COSMETIC FLUID 9109-OH

1-15%

### Andisil XL-12

0.25-12%

### KSG-320

1-5%

### ACULYN™ 88 Rheology Modifier

0.5-12%

### Andisil CE-13

1-15%

### Phenoxyethanol

0.5-1.5%

### CHEMSIL K-17

0.15-20%

### Dow Corning® 9041

1-15%

### Dow Corning® 2051

0.5-3%

### Dow Corning 9701

1-5%

### Licorice Extract

1-12%

### Astragulas Extract

1-15%

### BELSIL® CM 3092

1-10%

### Xiameter 200 100 cSt

5-20%

### VOLASIL™ DM-1

1-15%

### SP-10-FS

0.15-12%

### Platinum Catalyst

0.05-4.5%

### Matrixyl 3000

1-8%

### Glycerine 99.7%

1-18%

### 1,3 Butylene Glycol

1-17%

### Bronidox 1160

0.5-2.5%

### Kaolin Clay

0.15-12%

### Titanium Dioxide

0.05-7%

### Aluminum Dioxide

0.5-7%

### Zirconium Dioxide

0.05-9%

### F100-239-27-6

### KSP-300

1-18%

### GELAID 8205

0.5-15%

### COSMETIC FLUID 8110-DMC

0.5-8%

### Andisil CE13

0.25-12%

### Andisil XL-17

0.15-13%

### CARESIL™-XG CXG-1103

0.25-8%

### Nylon 10-12

1-7%

### Gransurf 61

0.25-15%
Graphene complex
0.05-9%

### Zeosil 142

0.15-12%

### KF-9901

1-10%

### Xiameter® Pmx-0345

1-20%

### VQM 199-200

0.05-12%

### KTZ® DIAMOND BRIGHT

0.15-5%

### Bronidox 1160

0.5-1.5%

### Titanium Dioxide

0.05-7%

### Aluminum Dioxide

0.5-7%

### Zirconium Dioxide

0.05-9%

### KoboGel™ D5 Medium

1-18%

### Mirasil C-DMX 12

0.5-12%

### BELSIL® CM 740

0.5-8%

### Dow Corning® 9509

0.5-17%

### CHEMSIL K-61

1-15%

### AEROSIL® R 812 S

0.5-13%

### Chitosan

0.05-8%

### X-21-5666

1-10%

### GI CD-965

0.5-20%

### Chrysanthemum Extract

1-15%

### Licorice Extract

1-18%

### Platinum Catalyst

0.15-18%

### CHEMSIL SPD

0.5-8%

### Carbopol 21

0.3-5%

### ACULYN™ 38 −0.15-7%

### Xiameter 245

1-15%

### COSMETIC FLUID 7007-DM

### Euxyl PE 9010

0.5-2%

### F100-245-37-7

### Dow Corning MG7-9900A

0.15-8%

### CHEMSIL K-17

0.5-12%

### Andiform C300

1-75%

### Granactive Oligopeptide-10

0.15-18%

### Andiform C106

1-29%

### Cabosil TS-500

0.5-12%

### USG-103

0.25-10%

### Andisil CE 500

0.05-18%

### Xiameter 1184

1-15%

### Andsil XL-11

0.15-12%

### DC TI-3011

0.05-12%

### Dow Corning 9509

1-15%

### Andisil SF 1.00

0.5-17%

### CARESIL™-XG CXG-1103

0.25-8%

### Andisil SF 0.65

0.1-20%

### Bronidox 1160

0.5-1.5%

### Gemtone Garnet G009

0.05-5%
Graphene complex
0.05-8%

### Titanium Dioxide

0.05-2.8%

### Zirconium Dioxide

0.25-3%

### Aluminum Dioxide

0.25-2%

### Dow Corning 9011

1-18%

### Xiameter 0245

1-15%

### Dow Corning 9546

1-18%

### Dow Corning MG7-9900B

0.5-5%

### Dow Corning 9041

1-17%

### Andisil SF 1.00

1-12%

### Andisil SF 0.65

0.5-18%

### Nylon 0-12

0.5-20%

### Magnesium Aluminum Silicate

0.01-8%

### Dow Corning 9509

5-15%

### Astragalus

0.5-10%

### Licorice

3-12%

### Platinum Catalyst

0.05-14%

### Solubilisant LRI

5-8%

### Chrysanthemum

1-10%

### Ginseng

1-12%

### Kaolin

2-6%

### Nephrite Extract

0.05-7%

### Plantacare 818UP

1-8%

### Glycerine 99.7%

1-12%

### 1,3 Butylene Glycol

1-17%

### Chitosan

0.01-5%

### GELAID® 8205

1-10%

### VQM 199-200

0.01-7%

### Propylene Glycol

1-13%

### Matrixyl 3000

1-5%

### Euxyl PE 9010

0.5-2.5%

### F100-248-65-8

### KSG-15

0.5-12%

### KF-9008

1-7%

### AEROSIL® R 812 S

1-10%

### CHEMSIL K-27

0.5-8%

### Mirasil CM5 HP

1-15%

### Dow Corning® 1515 Gum

1-8%

### VOLASIL™ 8515

2-10%

### Andisil XL-10

0.01-8.5%
Graphene complex
0.01-7%

### Andiform C100

1-35%

### Mica S-25

0.01-2.5%

### CARESIL™-XG CXG-1103

1-8%

### Xiameter 200

1-15%

### Titanium Dioxide

0.05-7%

### Aluminum Dioxide

0.5-7%

### Zirconium Dioxide

0.05-9%

### COSMETIC FLUID 7007-DM

1-20%

### KoboGel™ D5 Medium

1-15%

### HDK® H30

0.5-3%

### Glycerine 99.7%

1-12%

### Mica S-25

0.15-5%

### Licorice

1-12%

### Astragulas

### Platinum Catalyst

0.01-7.5%

### Nylon 10-12

1-8%

### Bronidox 1160

0.5-1.5%

### VOLASIL™ DM-1.5

1-15%

### Dow Corning® EP-9215 Cosmetic Powder

1-10%

### Xiameter 200

1-18%

### X-21-5666

1-15%

### Titanium Dioxide

0.05-7%

### Aluminum Dioxide

0.5-7%

### Zirconium Dioxide

0.05-9%

### F100-251-48-9

### BELSIL® RG 100

0.5-20%

### Dow Corning® 9546

1-18%

### Andisil® VS 1000

1-10%

### Granpowder Silica

3-7%

### Kaoline

0.5-7%

### Aristoflex HMB

0.25-5%
Graphene complex
0.05-12%

### Dow Corning® AP-8087 Fluid

1-15%

### Andisil XL-11

0.05-17%

### AEROSIL® R 106

1-10%

### SilCare Silicone 41M15

1-10%

### Gemtone Garnet G009

0.05-7%

### Euxyl PE 9010

0.5-2.5%

### Titanium Dioxide

0.05-7%

### Aluminum Dioxide

0.5-7%

### Zirconium Dioxide

0.05-9%

### CMF655T4*

0.5-15%

### BELSIL® REG 1102

1-10%

### Xiameter® Pmx-1501 Fluid

2-8%

### GI CD-10/11

1-15%

### EMULSIL® WO-5115

1-8%

### Nylon 10-12

0.5-12%

### Platinum Catalyst

0.05-7.5%

### Dow Corning® 1515 Gum

1-9%

### Propylene Glycol

1-17%

### Glycerine 99.7%

3-10%

### 1,3 Butylene Glycol

1-20%

### Chrysanthemum Extract

1-8%

### Astragulas Extract

2-12%

### Xiameter 200 10 cSt

1-15%

### Kaoline Clay

0.25-5%

### Dow Corning® Rm 2051

0.25-5%

### Titanium Dioxide

0.05-7%

### Aluminum Dioxide

0.5-7%

### Zirconium Dioxide

0.05-9%

### F100-255-32-10

### Dow Corning MG7-9900A

0.5-25%

### AEROSIL® R 106

0.5-8%

### Dow Corning® 3901

1-18%

### COSMETIC FLUID 9109-OH

1-20%
Graphene complex
0.05-12%

### USG-103

1-15%

### Andisil XL-17

0.05-10.5%

### Andisil CE-4

0.05-8%

### Xiameter 200 10 cSt

1-25%

### Titanium Dioxide

0.05-7%

### Aluminum Dioxide

0.5-7%

### Zirconium Dioxide

0.05-9%

### Dow Corning MG7-9900B

1-25%

### Dow Corning® 9045

5-22%

### SK-INFLUX® V

1-15%

### Dow Corning® Rm 2051

0.5-5%
Dow Corning® 670 fluid
1-15%

### Platinum Catalyst

0.05-12%

### Glycerine 99.7%

1-20%

### 1,3 Butylene Glycol

1-18%

### Licorice Extract

1-12%

### Jujube Extract

1-17%

### Granpowder Silica

1-10%

### Propylene Glycol

1-17%

### Bronidox 1160

0.5-2.5%

### Titanium Dioxide

0.05-7%

### Aluminum Dioxide

0.5-7%

### Zirconium Dioxide

0.05-9%

### F100-248-31-11

### Dow Corning MG7-9900A

1-15%
Graphene complex
3-5-12%

### Andiform C106

2.5-10%

### Dow Corning® 9040

3-15%

### Andisil XL-17

0.5-17%

### DC 9509

3-12%

### Xiameter 0244

1-15%

### Nephrite Powder Extract

0.05-8%

### Xiameter 345

1-18%

### Titanium Dioxide

0.2-9%

### Aluminum Dioxide

0.5-12%

### Zirconium Dioxide

0.2-8%

### Dow Corning MG7-9900B

1-12%

### Dow Corning® 9046

5-28%

### Dow Corning® 9509

0.5-12%

### Platinum C1142AF

1-8%

### Glycerine

5-10%

### Butylene Glycol

3-12%

### Nylon 10-12

0.5-10%

### Xiameter 200 5 cSt

1-18%

### Andisil 1 cSt

1-10%

### Astragulas Extract

5-12%

### VQM 199-200

0.05-5%

### Titanium Dioxide

0.05-7%

### Aluminum Dioxide

0.5-7%

### Zirconium Dioxide

0.05-9%

### F100-279-23-12

### Dow Corning® 9045

1-10%

### Cabosil TS-720

0.5-12%

### BOTANAGEN

1-8%

### CELL-U-LASH™ 90

1-5%

### Andisil C300

1-15%
Graphene complex
1-12%

### Dow Corning® 3901

3-12%

### Xiameter 200 100 cst

1-25%

### Xiameter 345

1-7%

### Umicore CX11

0.5-10%

### Mica S-25

0.05-3.5%

### Silk Peptides

1-10%

### CHEMSIL K-27

5-15%

### Titanium Dioxide

0.05-9%

### Aluminum Dioxide

0.5-5%

### Zirconium Dioxide

0.05-7%

### Dow Corning® 9011

1-8%

### Dow Corning® 9701

2-5%

### Glycerine

5-12%

### ELIGHT SDS

0.5-8%

### DL-PANTHENOL

1-8%

### Dow Corning® 9041

1-17%

### Zeosil 215GR

2-5%

### Propylene Glycol

1-15%

### Butylene Glycol

5-10%

### SP-10-FS

1-15%

### SunSpheres™ Powder

0.5-7%

### Umicore M22

2-6%

### Xiameter® Pmx-1411

1-12%

### L-ASCORBIC ACID USP

1-5%

### VITAMIN E

1-10%

### LICORICE EXTRACT

1-12%

### Titanium Dioxide

0.05-5%

### Aluminum Dioxide

0.5-8%

### Zirconium Dioxide

0.05-7%

### F100-321-75-13

### Dow Corning® 9509

3-15%

### Dow Corning® 3901

1-12%

### KoboGel™ D5 Medium

1-18%

### Dow Corning® 593

2-8%

### CHEMSIL K-51

3-16%

### Andsil XL-17

1-12%
Graphene complex
0.05-15.28%

### JEESILC PS-DMBF

1-10%

### Andisil CE13

1-8%

### Titanium Dioxide

0.05-3%

### Aluminum Dioxide

0.5-10%

### Zirconium Dioxide

0.05-5%

### Dow Corning® 9546

1-20%

### Dow Corning® 3901

0.5-15%

### Dow Corning® Pf-9510

1-7%

### Butylene Glycol

1-15%

### Dow Corning® 9040

3-12%

### Granpowder PSQ-Pt

1-7%

### Zinc Gluconate

1-8%

### Matrixyl 3000

2-7%

### Glycerine

1-15%

### Glycyrrhiza Uralensis (Licorice) Extract

2-8%

### Zizyphus Jujuba Fruit Extract

3-9%

### Propylene Glycol

2-18%

### Titanium Dioxide

0.05-12%

### Aluminum Dioxide

0.5-8%

### Zirconium Dioxide

0.05-7%

### F100-222-43-14

### Dow Corning® Pf-9510

3-12%

### Andisil® CE-4

0.5-8%

### Dow Corning® BY 11-030

1-9%

### JEESILC PS-CMBF

1-12%

### KSG-15

1-18%

### Andisil® XL-1B

### Cabosil TS-610

1-12%
Graphene complex
0.5-8%

### EMULSIL® WO-3115

1-7.5%

### Nephrite Powder Extract

0.05-3%

### SMS-1 SERICITE S

0.05-4.5%

### Euxyl PE 9010

1-5%

### Titanium Dioxide

0.05-11%

### Aluminum Dioxide

0.5-6%

### Zirconium Dioxide

0.05-5%

### Dow Corning® EL-9240

1-18%

### Dow Corning 9011

1-10%

### Dow Corning® CE 8401

1-15%

### Xiameter 245

1-17%

### Astragulas Membranaceus Root Extract

1-18%

### Zizyphus Jujuba Fruit Extract

2-15%

### Glycerine

5-18%

### Cabosil TS-510

1-8%

### Glycyrrhiza Uralensis (Licorice) Extract

1-13%

### Silk Amino Acid

2-5%

### Butylene Glycol

1-18%

### Silk Peptides

1-19%

### Platinum Catalyst

1-15%

### Tocopheryl Acetate

1-5%

### Zinc Gluconate

2-8%

### Bronidox 1160

1-5%

### Propylene Glycol

1-17%

### Titanium Dioxide

0.05-5.2%

### Aluminum Dioxide

0.5-10.8%

### Zirconium Dioxide

0.05-5.5%

### F100-287-18-15

### USG-103

1-20%

### Andisil® CE-500

1-5%

### JEESILC PS-DMLV

1-17%

### Xiameter 0.44

1-15%

### CHEMSIL K-61

1-12%

### SILICA SHELLS

1-11.5%
Graphene complex
0.5-17%

### MSS-500

0.05-3.8%

### HDK® V15

1-8%

### Dow Corning® Toray FZ-2222

1-8%

### Andisil® XL-17

1-7%

### Bronidox 1160

1-5%

### Gemtone® Garnet

0.05-5%

### EMULSIL® WO-3115

1-3.7%

### Titanium Dioxide

0.05-8.5%

### Aluminum Dioxide

0.5-12.5%

### Zirconium Dioxide

0.05-6.8%

### Dow Corning® 9040

1-20%

### Dow Corning® 1515 Gum

1-15%

### Dow Corning 9509

1-12%

### Zizyphus Jujuba Fruit Extract

1-18%

### Chrysanthemum Extract

1-10%

### Glycyrrhiza Uralensis (Licorice) Extract

1-20%

### Butylene Glycol

1-18%

### Glycerine

1-13%

### Platinum Catalyst

1-12%

### Dow Corning TI-3011

3-15%

### Xiameter® Pmx-0344

1-18%

### EcoSense™ 919

1-20%

### Xiameter 345

1-12%

### Bronidox 1160

1-8%

### Titanium Dioxide

0.05-11%

### Aluminum Dioxide

0.5-7.5%

### Zirconium Dioxide

0.05-8%

### Clinical Vitro Studies:

In clinical study, based upon 30 women tested, they use the transparent 2-step formulations only. The following 8 charts are the feedbacks from these 30-group of women after testing a sample product (17198—Beyond Flawless™ Second Skin; formula #F100-245-37-7).
Q1 After using this Beyond Flawless™ Second Skin, my eye bags disappeared almost instantaneously, within minutes

TABLE 1
Response from 30 test participants on eye bag reduction

| | |
|---|---|
| Answer Choices | Responses |

| | | |
|---|---|---|
| Strongly Agree | 70.00% | 21 |
| Agree | 30.00% | 9 |
| Disagree | 0.00% | 0 |
| Strongly Disagree | 0.00% | 0 |
| Total | | 30 |

Q2 After using this Beyond Flawless™ Second Skin, my eye dark circles disappeared immediately

TABLE 2
Response from 30 test participants on eye dark circle reduction

| | |
|---|---|
| Answer Choices | Responses |

| | | |
|---|---|---|
| Strongly Agree | 86.67% | 26 |
| Agree | 10.00% | 3 |
| Disagree | 3.33% | 1 |
| Strongly Disagree | 0.00% | 0 |
| Total | | 30 |

Q3 I feel comfortable wearing this Beyond Flawless™ Second Skin under my eyes, no allergic reaction

TABLE 3
Response from 30 test participants on allergy

| | |
|---|---|
| Answer Choices | Responses |

| | | |
|---|---|---|
| Strongly Agree | 100.00% | 30 |
| Agree | 0.00% | 0 |
| Disagree | 0.00% | 0 |
| Strongly Disagree | 0.00% | 0 |
| Total | | 30 |

Q4 I wear this Beyond Flawless™ Second Skin under my eyes for at least 24-48 hours, it is invisible and there are no edges peeling off

TABLE 4
Response from 30 test participants on product mechanic issue

| | |
|---|---|
| Answer Choices | Responses |

| | | |
|---|---|---|
| Strongly Agree | 80.00% | 24 |
| Agree | 20.00% | 6 |
| Disagree | 0.00% | 0 |
| Strongly Disagree | 0.00% | 0 |
| Total | | 30 |

Q5 I wear this Beyond Flawless™ Second Skin under my eyes during the day, and there are no issues after I encountered water.

TABLE 5
Response from 30 test participants on water bearing issue

| | |
|---|---|
| Answer Choices | Responses |

| | | |
|---|---|---|
| Strongly Agree | 50.00% | 15 |
| Agree | 50.00% | 15 |
| Disagree | 0.00% | 0 |
| Strongly Disagree | 0.00% | 0 |
| Total | | 30 |

Q6 It is easy to apply this Beyond Flawless™ Second Skin under my eyes. After drying, it is easy to apply make-up powder

TABLE 6
Response from 30 test participants on makeup application

| | |
|---|---|
| Answer Choices | Responses |

| | | |
|---|---|---|
| Strongly Agree | 86.67% | 26 |
| Agree | 10.00% | 3 |
| Disagree | 3.33% | 1 |
| Strongly Disagree | 0.00% | 0 |
| Total | | 30 |

Q7 After I apply this Beyond Flawless™ Second Skin under my eyes, I saw a reduction of my wrinkles and fine lines around my eyes

TABLE 7
Response from 30 test participants on reduction
of wrinkles & fine lines around eyes

| | |
|---|---|
| Answer Choices | Responses |

| | | |
|---|---|---|
| Strongly Agree | 50.00% | 15 |
| Agree | 40.00% | 12 |
| Disagree | 6.67% | 2 |
| Strongly Disagree | 3.33% | 1 |
| Total | | 30 |

Q8 After I take off this Beyond Flawless™ Second Skin under my eyes, I feel the skin under my eyes is much hydrated

TABLE 8
Response from 30 test participants on hydration issue

| | |
|---|---|
| Answer Choices | Responses |

| | | |
|---|---|---|
| Strongly Agree | 70.00% | 21 |
| Agree | 30.00% | 9 |
| Disagree | 0.00% | 0 |
| Strongly Disagree | 0.00% | 0 |
| Total | | 30 |

It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A kit for improving skin aesthetic, comprising
(a) a base component comprising graphene and a cross-linkable elastomer;
(b) an activating component comprising an agent promoting or catalyzing the cross-linking of the elastomer.

2. The kit of claim 1, wherein the graphene is characterized by a flake size ranging from about 0.5 to about 20 nm and a thickness ranging from about 0.5 to about 10 nm.

3. The kit of claim 1, wherein the graphene ranges from about 0.01% to about 30% by weight in the base component.

4. The kit of claim 1, wherein the graphene ranges from about 0.05% to about 20% by weight in the base component.

5. The kit of claim 1, wherein the base component further comprises a hydride functionalized polysiloxane selected from the group consisting of hydride terminated polydimethylsiloxane; hydride terminated polyphenyl-(dimethylhydrosiloxy)siloxane; hydride terminated methylhydrosiloxane-phenylmethylsiloxane copolymer, and bis vinyl terminated polysiloxane.

6. The kit of claim 1, wherein the base component is substantially free of water.

7. The kit of claim 1, wherein the cross-linkable elastomer is siloxane having a functional group for cross-linking.

8. The kit of claim 1, wherein the cross-linkable elastomer is vinyldimethicone.

9. The kit of claim 1, wherein the base component further comprises one or more agents selected from the group consisting of trisiloxane, disiloxane, dimethicone, and dimethiconol.

10. The kit of claim 1, wherein the base component and/or the activating component further comprises a pressure sensitive adhesive selected from the group consisting of ethenyldimethylsilyl oxy and trimethylsilyl oxy modified silica, polydimethylsiloxane gum and resin.

11. The kit of claim 1, wherein the agent for promoting cross-linking is a platinum catalyst.

12. The kit of claim 1, wherein the activating component further comprises one or more of trisiloxane, disiloxane, dimethicone, and dimethiconol.

13. The kit of claim 1, wherein the activating component further comprises a cross-linked vinyl dimethicone.

14. The kit of claim 1, further comprising in one or both of the base component and the activating component, one or more agents selected from the group consisting of feel modifiers, spreadability enhancers, adhesion modifiers, diluents, tack modifiers, optics modifiers, particles, volatile siloxanes, emulsifiers, emollients, surfactants, thickeners, solvents, film formers, humectants, preservatives and pigments.

15. The kit of claim 1, further comprising in one or both of the base component and the activating component, a reinforcing agent selected from the group consisting of mica, zinc oxide, titanium dioxide, aluminum oxide, clay, silica, fumed silica and multiple functional minerals.

16. The kit of claim 1, further comprising in one or both of the base component and the activating component, at least one of a cosmetic agent and a therapeutic agent.

17. The kit of claim 1, wherein the base component and the activating component are of translucent color or multiple tinted color.

18. A film for improving skin aesthetic, comprising graphene and cross-linked elastomer, wherein the graphene ranges from about 0.01% to about 30% by weight in the base component.

19. The film of claim 18, wherein the graphene is a single layer or multiple layers.

20. A method of improving skin aesthetic, comprising
(a) applying to skin the base component of claim 1; and
(b) applying to the skin the activating component of claim 1.

21. (canceled)

22. (canceled)

23. (canceled)
